(19) United States
(12) Patent Application Publication
(10) Pub. No.: US 2024/0415490 A1
(43) Pub. Date: Dec. 19, 2024

(54) ULTRASOUND COUPLING AGENT

(71) Applicant: SonoClear AS, Oslo (NO)

(72) Inventors: Wilhelm GLOMM, Oslo (NO); Geirmund UNSGÅRD, Oslo (NO); Tormod SELBEKK, Oslo (NO)

(21) Appl. No.: 18/712,472

(22) PCT Filed: Nov. 23, 2022
(86) PCT No.: PCT/EP2022/083056

§ 371 Date: May 22, 2024

(30) Foreign Application Priority Data
Nov. 23, 2021 (GB) 2116863.8

(51) Int. Cl. A61B 8/00

(52) U.S. Cl. CPC A61B 8/4281

(57) ABSTRACT

The present invention relates to aqueous ultrasound coupling agents comprising: a) 20.0 to 90.0 g/L of at least one triglyceride; b) 1.2 to 5.4 g/L of at least one phospholipid; c) optionally at least one humectant; and d) optionally at least one non-ionic stabilising agent. The invention also relates to the use of said aqueous ultrasound coupling agents in invasive ultrasound imaging of a subject. The invention also relates to methods of preparing said ultrasound coupling agents and methods of formulating said ultrasound coupling agents.

The present invention relates to ultrasound coupling agents and their use in invasive ultrasound imaging of a subject. The invention also relates to methods of preparing ultrasound coupling agents and methods of formulating ultrasound coupling agents.

### BACKGROUND

Ultrasound imaging is widely used in medical examination to obtain real-time visualization of tissue and is used in various clinical fields. To ensure proper contact between the transducer/ultrasound probe and the skin/tissue to be examined an ultrasound coupling agent, which is typically a gel or liquid, is used. The ultrasound coupling agent is used to avoid air pockets between the transducer/probe and tissue, and to facilitate a good acoustic coupling at the interface between the ultrasound transducer and the tissue to be imaged.

Ultrasound imaging is often used in invasive procedures, such as during surgery or during an invasive diagnostic procedure. For example, ultrasound imaging may be used for imaging of tumours in brain surgery. In this case, ultrasound imaging may be used to locate the tumour and anatomical structures, as well as to identify residual tumour during surgery. Ultrasound imaging may also be used to make sure that the damaged tissue, tumour etc. is completely removed, and that unnecessary resection of healthy tissue is avoided.

Ideally, high image quality should be sustained throughout the whole of a medical procedure in order to monitor the progress of that procedure (e.g. of surgery for tumour resection). However, the progress of procedures such as surgery may cause more image noise to be generated and thus lead to more inaccurate display of the relevant part of the anatomy in the ultrasound images.

The term artefact is used in medical imaging to describe any part of the image that does not accurately represent the anatomy of the subject being investigated, and it is well known that ultrasound imaging is prone to several different types of artefacts which may lead to adverse outcomes for the subject. For example, when using ultrasound in brain tumour surgery, the presence of artefacts may interfere with the surgeon's interpretation of the images.

One artefact commonly encountered when using ultrasound for the invasive imaging of the body is an enhanced (anomalously bright) signal appearing below a fluid-filled cavity. The brightness enhancement of tissue located beneath fluid filled spaces has been observed in ultrasound imaging of cysts, blood vessels or other fluid filled spaces. Because of this apparent enhancement of the reflected echo this frequently encountered image artefact is often referred to as a brightness artefact (sometimes also referred to as an “enhancement artefact”, “or “bright rim effect”). This type of artefact is a result of the difference in attenuation of acoustic waves by the fluid in the cavity and by the surrounding biological tissue.

For example, during brain tumour surgery a resection cavity is typically filled with saline water as an ultrasound coupling agent before ultrasound imaging, to enable propagation of sound and to prevent air artefacts. The difference in attenuation between brain and isotonic saline may cause artefacts that degrade the ultrasound images, potentially affecting surgical accuracy (e.g. resection grades and safety). The acoustic waves travel through the cavity filled with saline water before reaching the biological tissue. The attenuation of acoustic waves in saline water is very low compared to the attenuation of acoustic waves in biological tissue. The attenuation coefficient (a) for water is 0.0022 dB/(MHz·cm) while for e.g. brain it is reportedly measured by various groups to be within approximately 0.4-1.0 (Duck FA, In Physical properties of tissue, Academic Press, LTD).

A major component of the attenuation of sound in tissue is caused by absorption, in which part of the acoustic energy is converted to heat. Scattering also contributes to the attenuation of acoustic waves. These combined effects cause the acoustic waves propagating in saline water to have higher amplitudes than acoustic waves propagating an equal distance in biological tissue. The total attenuation is estimated by the equation:

Attenuation[dB]=α[dB/(MHz*cm)]*l[cm]*f[MHz]

Wherein α is the attenuation coefficient of the medium;
l is the medium length (or propagating distance); and
f is the frequency of the transmitted ultrasound wave.

For example, selecting a frequency of 8 MHz and a propagating distance of 10 cm, and assuming an attenuation coefficient of 0.8 for brain tissue this will result in an attenuation of 0.18 dB for ultrasound propagating in water and an attenuation of 64 dB for waves propagating in brain tissue.

This difference in attenuation can generate noise in the ultrasound images e.g. when ultrasound is used intraoperatively in brain tumour surgery. The ultrasound waves transmitted through the water-filled resection cavity will have a large amplitude when arriving at the cavity walls, due to the low attenuation of water. Thus, the sound waves reflected from the cavity wall will also have relatively high amplitudes, see FIG. 1. Further, the sound waves propagating further into the tissue will have relatively high amplitudes. In comparison to sound waves that have propagated entirely in brain tissue, with a relatively high attenuation coefficient, these transmitted and reflected waves that have travelled through saline will be less damped and thereby have significantly higher amplitudes. The fluctuations in intensity observed in the ultrasound images make it very difficult to interpret the images, (see FIG. 1). The bright rim observed at the cavity wall may mask the presence of residual tumour, or the high intensity regions extending from the cavity wall may be interpreted as hyperechoic tumour when it is actually normal brain tissue.

The presence of the hyperechoic rim in ultrasound imaging of a resection cavity is described in several papers regarding the use of ultrasound in brain tumour surgery. This enhanced signal appearing below the fluid filled cavity in the ultrasound images is regarded as one of the major imaging artefacts encountered in peroperative ultrasound imaging.

WO 2013/167654A1 relates to an aqueous ultrasound coupling fluid comprising a pharmaceutical grade triglyceride and a pharmaceutically acceptable emulsifier. In the Examples of this document, the ultrasound coupling fluid is used to image both a phantom and fresh piglet cadavers. In both cases, the use of an ultrasound coupling fluid according to that invention is shown to reduce the appearance of brightness artefacts when compared to the use of saline as an ultrasound coupling agent. However, there is no experimental data on the performance of the ultrasound coupling agent in humans nor in relation to the stability of the material to sterilisation of storage.

In the course of their research leading up to the development of the present invention, the present inventors unexpectedly established that the ultrasound coupling agent exemplified in WO 2013/167654A1 did not perform as well in reducing the appearance of brightness artefacts during the ultrasound imaging of human tissue as might otherwise be expected based on the performance reported in the phantom and fresh piglet cadavers. Furthermore, the performance in relation to sterilisation and storage may be improved.

In addition, the inventors established that the use of (pure) sterile water (i.e. a non-isotonic, non-ionic solvent) to prepare the aqueous ultrasound coupling agents led to potential safety concerns and was not suitable for certain application such as the imaging of the brain. To solve this problem, an isotonic solvent such as isotonic saline can be used to prepare the aqueous ultrasound coupling agent. However, the inventors have observed that the use of saline in the aqueous ultrasound coupling agent leads to the separation of the fat emulsion phase. This phase-separation may be observed with or without autoclaving, but particularly after sterilisation in this way. It would evidently be an advantage to provide ultrasound coupling agents which are more stable to storage and/or sterilisation, particularly with regard to phase-separation.

Moreover, in order to sterilise an ultrasound coupling agent comprising an isotonic solvent such as saline, typically one has to irradiate the composition e.g. using gamma radiation. However, the use of gamma radiation to sterilise the ultrasound contact agents has been observed to lead to a drop in pH. The size of the drop in pH varies depending on the radiation dose. The change in pH may be unpredictable and therefore difficult to mitigate against. The use of an isotonic solvent such as saline to prepare the composition and subsequent sterilisation can therefore lead to the production of ultrasound coupling agents with variation in pH. It would be beneficial to have an ultrasound coupling agent which not only has better stability in the presence of ions, but also which shows less variance in pH change after irradiation.

The present inventors thus set out to provide further improved ultrasound coupling agents capable of reducing the appearance of artefacts in ultrasound images produced during invasive ultrasound imaging of a subject, particularly a human subject. The inventors further set out to provide ultrasound coupling agents which are better suited for reducing the appearance of artefacts in invasive ultrasound imaging of specific types of tissue, such as brain, heart, and breast tissue.

The present inventors have now surprisingly established that an aqueous ultrasound coupling agent comprising 20.0 to 90.0 g/L of at least one triglyceride and 1.2 to 5.4 g/L of at least one phospholipid provides excellent reduction in the appearance of artefacts, such as brightness artefacts, generated during invasive ultrasound imaging, particularly in humans.

Without wishing to be bound by theory, it is believed that the above amounts of triglyceride and phospholipid provide the aqueous coupling agent with an attenuation which is a better match for that of tissues found in the body (particularly the human body). The use of an aqueous ultrasound coupling agent according to the present invention therefore reduces the difference in signal intensity of reflected waves which have propagated primarily in the fluid and those which have propagated primarily in the tissue. In addition, by formulating the aqueous ultrasound coupling agent using the amounts of triglyceride and phospholipid described above, excellent physical properties (e.g. in terms of homogeneity, droplet size and droplet size distribution) may be obtained.

In addition, the inventors have surprisingly established that the addition of a non-ionic stabilising agent to the aqueous coupling agent may improve the stability of the ultrasound coupling agent. This may be especially where there is a high (e.g. isotonic) concentration of ionic species present in the aqueous portion of the composition e.g. resulting from the use of saline to prepare the composition. Moreover, the addition of a non-ionic stabilising agent may reduce the variance in pH after exposure of the acoustic coupling agent to gamma-radiation i.e. leading to a more predictable drop in pH.

The present inventors have also established particular compositions of aqueous ultrasound coupling agents which are better-suited for the invasive ultrasound imaging of specific tissue types, such as brain tissue, heart tissue and breast tissue.

### SUMMARY OF THE INVENTION

In one aspect, the invention provides an aqueous ultrasound coupling agent comprising:
a) 20.0 to 90.0 g/L of at least one triglyceride;
b) 1.2 to 5.4 g/L of at least one phospholipid;
c) optionally at least one humectant; and
d) optionally at least one non-ionic stabilising agent.

In another aspect, the invention provides an aqueous ultrasound coupling agent according to any aspect described herein for use in invasive ultrasound imaging of a subject, such as during surgery or during an invasive diagnostic procedure.

In another aspect, the invention provides an aqueous ultrasound coupling agent comprising:
a) 20.0 to 90.0 g/L of at least one triglyceride;
b) 1.2 to 5.4 g/L of at least one phospholipid;
c) optionally at least one humectant; and
d) optionally at least one non-ionic stabilising agent;
for use in invasive ultrasound imaging of the heart, such as during open-heart surgery, and wherein the attenuation coefficient (α) of the ultrasound coupling agent is in the range of 0.1 to 1.0 dB/(MHz·cm).

In another aspect, the invention provides an aqueous ultrasound coupling agent comprising:
a) 20.0 to 90.0 g/L of at least one triglyceride;
b) 1.2 to 5.4 g/L of at least one phospholipid;
c) optionally at least one humectant; and
d) optionally at least one non-ionic stabilising agent;
for use in invasive ultrasound imaging of the breast, such as during breast cancer surgery, wherein the attenuation coefficient (α) of the agent is in the range of 0.1 to 1.2 dB/(MHz·cm).

In all aspects and embodiments of the invention, the aqueous ultrasound coupling agent may be formulated/diluted to an appropriate concentration with an aqueous fluid to provide the concentrations described herein. This may be water (e.g. deionised water or water for injection). The aqueous fluid may additionally comprise at least one salt, such as at least one biocompatible salt (e.g. an alkali metal salt such as a sodium and/or potassium salt). In one embodiment, the aqueous component of the coupling agent will comprise around 0.1 to 1.0% of an biocompatible salt such as sodium chloride and/or potassium chloride.

In another aspect, the invention provides an aqueous ultrasound coupling agent according as hereinbefore defined for use in invasive ultrasound imaging of the colon in colorectal cancer staging using a balloon stand-off system, wherein the attenuation coefficient (α) of the ultrasound coupling agent is in the range of 0.1 to 0.6 dB(MHz·cm).

In another aspect, the invention provides a method of preparing an aqueous ultrasound coupling agent according to any aspect described herein, or an aqueous ultrasound coupling agent for use according to any aspect described herein, the method comprising:
i) providing a mixture comprising at least one triglyceride, at least one phospholipid and optionally at least one humectant; and
ii) diluting said mixture with water or an aqueous solution comprising water and optionally at least one non-ionic stabilising agent.

In another aspect, the invention provides a method of formulating an aqueous ultrasound coupling agent according to any aspect described herein, or an aqueous ultrasound coupling agent for use according to any aspect described herein, the method comprising:
i) providing a mixture comprising at least one triglyceride, at least one phospholipid and optionally at least one humectant;
ii) determining the attenuation coefficient (α) of the mixture;
iii) calculating the difference (d) between the attenuation coefficient of the mixture determined in step ii) and a reference attenuation coefficient (αᵣ); iv) forming a diluted mixture by diluting the mixture provided in step i) with water or an aqueous solution comprising water and optionally at least one non-ionic stabilising agent, in an amount which is determined by a function f(d) of the difference (d) calculated in step iii); and
v) optionally repeating steps ii)-iv) on the diluted mixture obtained in step iv).

In another aspect, the invention provides a kit comprising:
A) a mixture comprising at least one triglyceride, at least one phospholipid and optionally at least one humectant; and
B) water or an aqueous solution comprising water and optionally at least one non-ionic stabilising agent.

### BRIEF DESCRIPTION OF THE FIGURES

FIG. 1 shows reformatted image slices from 3D ultrasound volumes acquired intraoperatively as displayed on a navigation system. In the left image we observe a high intensity region at the bottom left cavity wall, introduced by waves transmitted through the low attenuation water filled cavity. In the right image the position of the ultrasound probe has been changed to the left of the operating channel, and we observe that the bright rim, or the apparent enhanced echo, is now observed in the bottom right cavity wall.

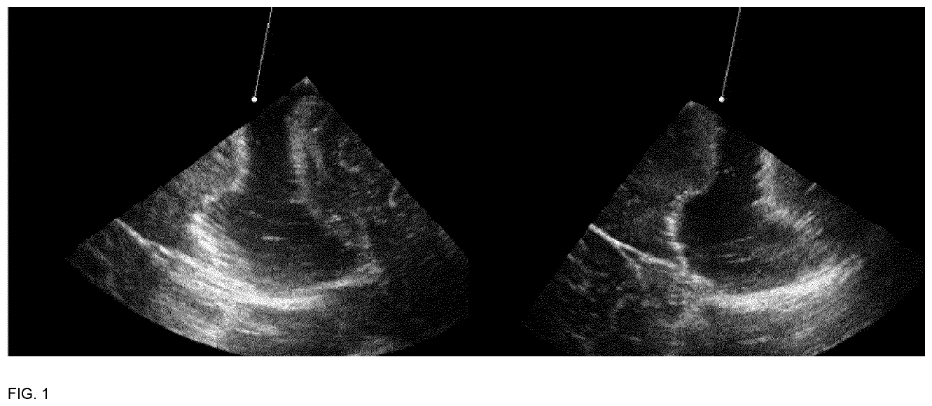

FIG. 2. (A) Ultrasound image of brain tissue using saline as ultrasound coupling agent. There is a bright rim apparent at the top cavity wall. (B) Ultrasound image of brain tissue using ultrasound contact agent of the present invention. Residual tumour is easily identified.

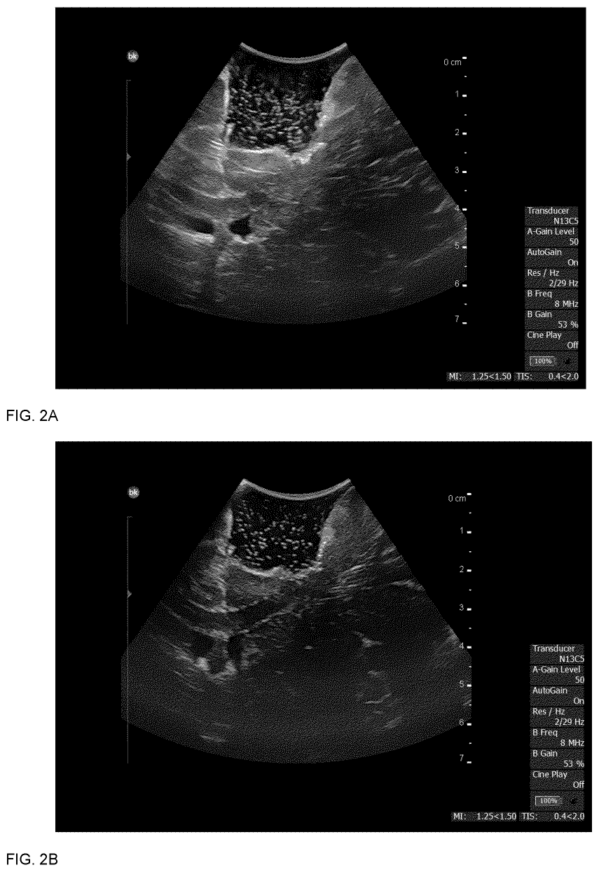

FIG. 3. (A) Ultrasound screening of colorectal wall using saline as ultrasound coupling agent. The cell layers are not easily identified. (B) Ultrasound screening of colorectal wall using ultrasound coupling agent of the present invention. There is clear cell layer delineation. This allows one to see any tumour cell invasion.

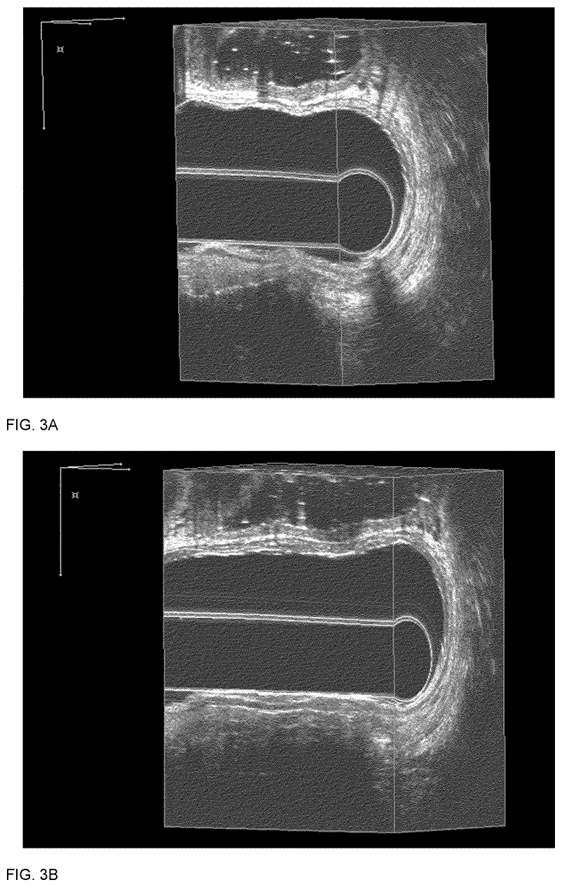

FIG. 4—Microscope image of Acoustic Coupling Agent without stabilising agent: 17.5 ml Lipid Concentrate (see Examples)+32.5 ml saline dilution −pH adjusted with NaOH

FIG. 5—Microscope image of Acoustic Coupling Agent with stabilising agent: 17.5 ml Lipid Concentrate (see Examples)+32.5 ml saline dilution containing 10 ml per 1000 ml polysorbate 80 (Tween 80) −pH adjusted with NaOH.

FIG. 6—Two views (A & B) of the acoustic coupling agent without stabilising agent, stored in a syringe at 40° C. Phase separation is visible.

### DETAILED DESCRIPTION

The present invention relates to an ultrasound coupling agent and the use thereof in invasive ultrasound imaging of a subject. The ultrasound coupling agent according to the present invention is unexpectedly effective in reducing the appearance of artefacts such as brightening artefacts when used in the invasive ultrasound imaging of body tissues, particularly human body tissues.

As used herein, the term “ultrasound coupling agent” refers to a medium used to exclude air between the ultrasound probe and the tissue to be imaged. The main purpose of the ultrasound coupling agent is thus to provide acoustic coupling between the ultrasound transducer/probe and the tissue to be investigated. The term is synonymous with terms like “contact fluid”, “contact agent”, “coupling agent”, “coupling fluid”, “acoustic fluid”, “acoustic coupling fluid” and “acoustic coupling agent”. In one embodiment, the ultrasound coupling agent described herein is in the form of a liquid or gel under standard conditions of temperature and pressure and/or at physiological temperatures and ambient pressure. Typically the ultrasound coupling agent is in the form of an emulsion.

The term “aqueous” as used herein refers to a solution or mixture comprising water as the solvent or continuous phase. This includes cases where an aqueous phase can diffuse freely within the confines of a free-standing three-dimensional structure such as a hydrogel or an emulsion-based hydrogel (e.g. an emulgel). The term does not exclude solvents other than water from being present in the solution/mixture, however typically water is the principal solvent present (i.e. forming at least 50% by weight of the solvent present in the solution/mixture). In a preferred embodiment, the term “aqueous” refers to a solution or mixture comprising water as the solvent or continuous phase, and in which water forms at least 90% by weight of the solvent (or continuous phase) present in the solution/mixture. In one embodiment, water is the only solvent present in the solution/mixture (or continuous phase). Aqueous solutions or mixtures may contain salts such as sodium and/or potassium salts and may, for example be isotonic. Aqueous solutions or mixtures may be sterile, as appropriate.

In one aspect, the aqueous ultrasound coupling agent is for use in invasive ultrasound imaging of a subject. As used herein, the term “invasive ultrasound imaging” refers to any method for imaging of a body, particularly the human body, which involves the detection of ultrasound and which involves the introduction of a device (such as the tip of an ultrasonic probe/transducer) into the body either through a body orifice or through an opening in the body other than a body orifice, such as a break or rupture in the skin. The term “body orifice” means any natural opening in the body, as well as the external surface of the eyeball, or any permanent artificial opening, such as a stoma. Invasive ultrasound imaging thus includes both the imaging of a body via the introduction of a device through a natural opening in the body and the imaging of a body via the introduction of a device through an artificial opening, such an incision made during surgery or as part of an invasive diagnostic procedure.

As used herein, the term “ultrasound” refers to sound waves with frequencies higher than the upper audible limit of human hearing. Typically, this is about 20 kHz and so in general ultrasound waves have a frequency greater than 20 kHz. In the applications envisaged in the present invention however, frequencies in the range of about 5 to about 20 MHz are contemplated. Preferably therefore, the term “ultrasound imaging” as used herein refers to any method for imaging involving the detection of sound waves having frequencies in the range of 5 to 20 MHz. “Imaging” may involve the display of a static and/or moving visual image, such as on paper or a display screen, or may involve a computer or artificial intelligence device generating and/or processing data that could be rendered as a visual image, whether or not such an image is in fact generated.

### Component a) (Triglyceride)

The aqueous ultrasound coupling agent according to the present invention comprises 20.0 to 90.0 g/L of at least one triglyceride. As used herein, the term “triglyceride” (sometimes also “triacylglycerol” or “triacylglyceride”) refers to a neutral tri-ester derived from glycerol and three fatty acids. The triglyceride as defined herein therefore consists of a polar glycerol-derived “head group” and three non-polar fatty-acid derived “tail groups”. The three non-polar tail groups may have the same or a differing number of carbon atoms and may each independently be saturated or unsaturated. It is preferred however that at least one of the three non-polar tail groups is unsaturated.

Examples of suitable non-polar groups include C6-C32 alkyl and alkenyl groups, which are typically present as the esters of long chain carboxylic acids. These are often described by reference to the number of carbon atoms and the number of unsaturations in the carbon chain. Thus, CX:Z indicates a hydrocarbon chain having X carbon atoms and Z unsaturations. Examples particularly include lauroyl (C12:0), myristoyl (C14:0), palmitoyl (C16:0), phytanoyl (C16:0), palmitoleoyl (C16:1), stearoyl (C18:0), oleoyl (C18:1), elaidoyl (C18:1), linoleoyl (C18:2), linolenoyl (C18:3), arachidonoyl (C20:4), behenoyl (C22:0) and lignoceroyl (C24:9) groups. Thus, typical non-polar chains are based on the fatty acids of natural ester lipids, including caproic, caprylic, capric, lauric, myristic, palmitic, phytanic, palmitolic, stearic, oleic, elaidic, linoleic, linolenic, arachidonic, behenic or lignoceric acids, or the corresponding alcohols. Preferable non-polar chains are palmitic, stearic, oleic and linoleic acids, particularly linoleic acid. In one preferred embodiment, component a) comprises at least one triglyceride having one or more C16 to C18 alkyl groups, particularly such groups having zero, one or two unsaturations. In particular, component a) may comprise at least 50% of triglycerides having such alkyl groups.

The at least one triglyceride used as part of component a) may be synthetic or may be derived from a purified and/or chemically modified natural source. In one particularly preferred embodiment, the at least one triglyceride is derived from a natural source. For example, the triglyceride used may be selected from vegetable oils or animal fats. Vegetable oils may preferably be selected from the group consisting of soybean oil, olive oil, palm oil and copra oil. Examples of animal fats that may be used are milk fats, fish oils and fish liver oils. The use of soybean oil (which is typically a mixture of neutral triglycerides) is especially preferred.

Component a) is present in the ultrasound coupling agent in an amount of 20.0 to 90.0 g/L. In a preferred embodiment, component a) is present in an amount of 40.0 to 90.0 g/L, more preferably 50.0 to 90.0 g/L. In an especially preferred embodiment, component a) is present in an amount of 55.0 to 85.0 g/L, most preferably 60.0 to 80.0 g/L. Where a mixture of triglycerides is used as component a), the amount (g/L) of component a) refers to the sum of the amount of each of the triglycerides in the composition as a whole.

When using triglycerides obtained from commercial sources, particular natural products such as soybean oil, there is generally a certain proportion of “contaminant” lipid having other chain lengths etc. In one aspect, component a) as defined herein may be any commercial grade of triglyceride with concomitant impurities (i.e. a triglyceride of commercial purity). These impurities may be separated and removed by purification but providing the grade is consistent this is rarely necessary. If necessary, however, component a) may be essentially chemically pure triglyceride, such as at least 80% pure, preferably at least 85% pure and more preferably at least 90% pure triglyceride.

Given that the aqueous ultrasound coupling agent is intended for use on the body, it will be readily appreciated by the skilled person in the art that certain additional requirements must also typically be satisfied by the at least one triglyceride. In particular, the at least one triglyceride and its component fatty acids must generally be non-toxic, sterile and biocompatible. In a preferred embodiment therefore, the at least one triglyceride is at least one pharmaceutical grade triglyceride.

### Component b) (Phospholipid)

Component b) of the aqueous ultrasound coupling agent is at least one phospholipid. This component comprises a polar head group and at least one non-polar tail group. The difference between components a) and b) lies principally in the polar group. The non-polar portions may thus suitably be derived from the fatty acids or corresponding alcohols considered above for component i). In particular C16 to C18 acyl groups having zero, one or two unsaturations are highly suitable as moieties forming the non-polar group of the compounds of component b). It will typically be the case that the phospholipid will contain two non-polar groups, although one or more constituents of this component may have only one non-polar moiety. Where more than one non-polar group is present these may be the same or different.

The at least one phospholipid of component b) comprises a negatively charged phosphate group. The phospholipid is thus an ionic species. Preferred phospholipid polar “head” groups include phosphatidylcholine (PC), phosphatidylethanolamine (PE), phosphatidylserine (PS) and phosphatidylinositol (PI). PC and PE are preferred lipids, both individually and as a mixture. In one embodiment, component b) may comprise at least 70% PC, PE or mixtures thereof. Most preferred is phosphatidylcholine (PC). In a preferred embodiment, component b) thus comprises at least 50% PC, preferably at least 70% PC and most preferably at least 80% PC.

The phospholipid component b) may be synthetic or derived from a natural source. In a preferred embodiment, component b) is derived from a natural source. Suitable natural sources of phospholipids include egg, heart (e.g. bovine), brain, liver (e.g. bovine) and plant sources including soybean. Such sources may provide one or more constituents of component b), which may comprise any mixture of phospholipids. In a particularly preferred embodiment, component b) is lecithin, preferably egg yolk lecithin, soy lecithin or a mixture thereof. Egg yolk lecithin is especially preferred. As for component a), the at least one phospholipid is typically non-toxic, biocompatible and sterile. Preferably therefore the at least one phospholipid is any pharmaceutically acceptable phospholipid.

In a preferred embodiment, component b) is present in an amount of 2.5 to 5.2 g/L of the aqueous ultrasound coupling agent, preferably 3.0 to 5.0 g/L. In a particularly preferred embodiment, component b) is present in an amount of 3.5 to 4.8 g/L, most preferably 3.8 to 4.5 g/L. Where component b) comprises a mixture of phospholipids, the amount component b) refers to the sum of the amount of each of the phospholipids in the composition as a whole. Component b) may contain low levels of impurities (i.e. lipids other than phospholipids) or if desired may be substantially pure.

Together with the triglyceride component a), component b) typically forms an emulsion when added to a suitable solvent. In the aqueous ultrasound coupling agent of the present invention, component a) and component b) preferably together form an oil-in-water (o/w) type emulsion i.e. wherein droplets of oil (comprising at least component a)) are dispersed within a continuous aqueous phase. The amphiphilic phospholipid component b) helps to stabilise the interface formed between the dispersed triglyceride/oil phase and the continuous aqueous phase.

The droplet size of the emulsion can be tuned by varying the nature and/or relative amount of triglyceride and phospholipid components (as well as any optional co-emulsifier(s) that may be present). Additionally or alternatively, the droplet size can be tuned by controlling the shear forces applied during emulsification.

Typically the number average (mean) droplet diameter is in the range of 100 to 1000 nm, preferably in the range of 200 to 700 nm, most preferably in the range of 230 to 340 nm. In an especially preferred embodiment, the number average droplet diameter is in the range of 250 to 320 nm, such as about 285 nm±35 nm, preferably 285 nm±5 nm. Average droplet diameters is measured by Dynamic Light Scattering using a Malvern Zetasizer.

In one embodiment the droplets in the emulsion are monodisperse or essentially monodisperse. As used herein, the term “monodisperse” means that at least 90% by number of the droplets (D90) have a diameter in the range of 200 to 400 nm. In a preferred embodiment the D90 of the emulsion is in the range of 220 to 350 nm, most preferably in the range of 230 to 340 nm.

In one embodiment, the ratio by weight between component a) and component b) in the aqueous ultrasound coupling agent is in the range of 8:1 to 25:1, preferably 10:1 to 22:1, more preferably 12:1 to 20:1, most preferably 14:1 to 18:1.

In a preferred embodiment, the aqueous ultrasound coupling agent according to the present invention comprises component b) as defined herein in combination with any of the preferred embodiments defined herein for component a).

For example, in one preferred embodiment, the aqueous ultrasound coupling agent comprises:
a) 60.0 to 80.0 g/L of at least one triglyceride; and
b) 3.8 to 4.5 g/L of at least one phospholipid.

In another preferred embodiment, the aqueous ultrasound coupling agent comprises:
a) 20.0 to 90.0 g/L soybean oil; and
b) 1.2 to 5.4 g/L lecithin.

In a particularly preferred embodiment, the aqueous ultrasound coupling agent comprises:
a) 60.0 to 80.0 g/L soybean oil; and
b) 3.8 to 4.5 g/L lecithin.

In a preferred embodiment, the lecithin is egg yolk lecithin, soy lecithin or a mixture thereof.

### Component c) (Humectant)

Optionally, the aqueous ultrasound coupling agent according to the present invention further comprises at least one humectant. As used herein, the term “humectant” refers to a hygroscopic substance which is capable of controlling the water content within a material. Humectants typically therefore comprise molecules having one or more hydrophilic groups, such as hydroxyl groups. Polyols are particularly preferred.

In a preferred embodiment, the aqueous ultrasound coupling agent comprises at least one humectant. The humectant is typically biocompatible, sterile, and non-toxic. In general therefore, any pharmaceutically acceptable humectant may be used. In a preferred embodiment, the at least one humectant is selected from the group consisting of polyols, lactic acid and mixtures thereof, especially polyols.

Example of polyols include glycerol (also known as glycerin/glycerine), propylene glycol, and sorbitol. The use of glycerol is especially preferred.

Where present, the humectant is typically present in the aqueous ultrasound coupling agent in an amount of 2.2 to 9.5 g/L, preferably 4.5 to 9.2 g/L, more preferably 5.5 to 9.0 g/L, most preferably 6.5 to 8.5 g/L. Where a mixture of humectants is used, the amount of humectant refers to the sum of the amount of each humectant present in the aqueous ultrasound coupling agent. As for all components of the aqueous ultrasound coupling agent of the present invention, the humectant is preferably biocompatible, sterile and non-toxic.

The humectant may contribute to controlling the osmotic pressure of the acoustic coupling agent, as described herein.

In one preferred embodiment, the aqueous ultrasound coupling agent comprises: a) 60.0 to 80.0 g/L of at least one triglyceride;
b) 3.8 to 4.5 g/L of at least one phospholipid; and
c) 6.5 to 8.5 g/L of at least one humectant.

In another preferred embodiment, the aqueous ultrasound coupling agent comprises:
a) 20.0 to 90.0 g/L soybean oil;
b) 1.2 to 5.4 g/L lecithin; and
c) 2.2 to 9.5 g/L glycerol.

In a particularly preferred embodiment, the aqueous ultrasound coupling agent comprises:
a) 60.0 to 80.0 g/L soybean oil;
b) 3.8 to 4.5 g/L lecithin; and
c) 6.5 to 8.5 g/L glycerol.

In a preferred embodiment the lecithin is egg yolk lecithin, soy lecithin or a mixture thereof.

### Component d) (Stabilising Agent)

In one aspect, the aqueous ultrasound coupling agent optionally further comprises at least one non-ionic stabilising agent. The purpose of this component, when present, is to aid in the creation and stabilisation of the emulsion formed within the aqueous ultrasound coupling agent. In this respect, the stabilising agent is similar to the phospholipid component b), however unlike the phospholipid the stabilising agent is non-ionic i.e. it does not comprise any charged groups. In addition, the phospholipid is intercalated into the droplet interface, whereas a stabilising agent does not necessarily have direct coupling with the droplet interface. Advantageously, the presence of at least one non-ionic stabilising agent in the ultrasound coupling agent improves the storage stability of the ultrasound coupling agent and prevents or retards phase separation of the oil and water phases.

Also, the addition of a non-ionic stabilising agent to the ultrasound coupling agent surprisingly increases the stability of the composition in the presence of dissolved ions, such as in the case where saline is used as the solvent used to prepare the aqueous phase of the composition. Whereas it was observed that an aqueous coupling agent prepared using saline led to separation of the composition after autoclaving, the inventors surprisingly established that the addition of a non-ionic stabilising agent such as polysorbate 80 to the aqueous coupling agent effectively prevented this phase separation. Adding the non-ionic stabilising agent also improved the stability of pH (less variance in measurements), especially after gamma irradiation.

In a preferred aspect of the invention therefore, the aqueous ultrasound coupling agent comprises:
a) 20.0 to 90.0 g/L of at least one triglyceride;
b) 1.2 to 5.4 g/L of at least one phospholipid;
c) optionally at least one humectant; and
d) at least one non-ionic stabilising agent (e.g. 0.5 to 20 g/L, such as 1 to 10 g/L).

In general, any non-ionic compound capable of aiding in the creation and stabilisation of the water (i.e. the oil-in water emulsion) may be used as the stabilising agent. Preferably however the stabilising agent is water-soluble as this aids in the creation of an oil-in-water (o/w) emulsion as opposed to a water-in-oil (w/o) emulsion. An additional advantage of using a water-soluble stabilising agent is that it is conveniently added directly to the aqueous ultrasound coupling agent or an aqueous solution used to form the aqueous ultrasound coupling agent. In a preferred embodiment, the non-ionic stabilising agent is a non-ionic surfactant.

In one embodiment, the non-ionic stabilising agent has a hydrophilic-lipophilic balance (HLB) value in the range of 8-14. The HLB value of a compound (given as a number on the scale of 0 to 20) is a measure of the degree to which it is hydrophilic or lipophilic and may be determined according to the method developed by Griffin (see W. C. Griffin, J. Soc. Cosmet. Chem. 1 (1949) 311; W. C. Griffin, J. Soc. Cosmet. Chem. 5 (1954) 249). An HLB value of 0 corresponds to a completely lipophilic/hydrophobic molecule, and a value of 20 corresponds to a completely hydrophilic/lipophobic molecule. Non-ionic stabilising agents having an HLB value in the range of 8-14 are particularly suitable for forming oil-in water emulsions and are thus preferred. In one embodiment, a mixture of non-ionic stabilising agents having an average HLB value in the range of 8-14 is used for optimal stability.

In a preferred embodiment, the non-ionic stabilising agent is a polyoxyalkylene (e.g. polyoxyethylene)-based stabilising agent. In one embodiment, said polyoxyethylene-based stabilising agents may comprise the repeat unit:

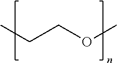

wherein n is an integer in the range of 2 to 130.

Such polyoxyalkylene (e.g. polyoxyethylene) groups may be attached to a surfactant or lipid molecule having a polar (non-ionic) head group and at least one non-polar tail group. Head groups include polyols such as sugars, glycerol, sorbitan etc. Tail groups include C10 to C22 (especially C16 to C20) fatty acyl groups (e.g. with zero, one, two, three or four unsaturations) such as oleate, linoleate, palmitolate, pinoleate etc. Each stabilising agent molecule may have 0-3, preferably 1 head group, 0-3, preferably 1 tail group and independently 1-5, preferably 2-4 polyoxyalkylene groups (see above). Polyoxyalkylene groups may alternatively or additionally be formed as part of a block copolymer.

In one embodiment, the non-ionic stabilising agent is selected from the group consisting of polysorbates, poloxamers and mixtures thereof. Examples of suitable polysorbates include polysorbate 80, polysorbate 60, polysorbate 40, polysorbate 20 and mixtures thereof. Examples of poloxamers (i.e. non-ionic triblock copolymers composed of a central hydrophobic change of polyoxypropylene flanked by two hydrophilic chains of polyoxyethylene) suitable for use in the present invention include those commercially available under the series name Pluronic® (e.g. Pluronic® F68) and Kolliphor® (both available from BASF) and Synperonic® (from Croda). The use of polysorbate 80 (P80—Polyoxyethylene (20) sorbitan monooleate) as a stabilising agent is especially preferred.

When present, component d) may be present in an amount of 2.0 to 9.0 g/L, preferably 4.0 to 8.8 g/L, more preferably 6.0 to 8.5 g/L, most preferably 6.0 to 8.0 g/L. Where component d) comprises a mixture of non-ionic stabilising agents, the amount of component d) refers to the sum of the amount of each humectant present in the aqueous ultrasound coupling agent.

In one preferred embodiment, the aqueous ultrasound coupling agent comprises: a) 60.0 to 80.0 g/L of at least one triglyceride;
b) 3.8 to 4.5 g/L of at least one phospholipid*;
c) 6.5 to 8.5 g/L of at least one humectant; and
d) 6.0 to 8.0 g/L of at least one non-ionic stabilising agent.

In another preferred embodiment, the aqueous ultrasound coupling agent comprises:
a) 20.0 to 90.0 g/L soybean oil;
b) 1.2 to 5.4 g/L lecithin*;
c) 2.2 to 9.5 g/L glycerol; and
d) 2.0 to 9.0 g/L polysorbate 80.

In a particularly preferred embodiment, the aqueous ultrasound coupling agent comprises:
a) 60.0 to 80.0 g/L soybean oil;
b) 3.8 to 4.5 g/L lecithin*;
c) 6.5 to 8.5 g/L glycerol; and
d) 6.0 to 8.0 g/L polysorbate 80.

*In a preferred embodiment the phospholipid/lecithin is egg yolk lecithin, soy lecithin or a mixture thereof.

Because the coupling agent is designed for use in contact with body tissue, a neutral pH is desirable. Tests (see Examples below) suggest that the decrease in pH observed on storage and/or irradiation of the ultrasound coupling agent can be significant. This can, of course, be compensated for by altering the initial pH of the formulations providing that pH change is predictable. The presence of the stabilising agent has been observed to reduce the variation in pH change upon irradiation and/or storage and therefore may allow for more accurate compensation for pH change and thus a more reliable product.

### Optional Further Additives

In addition to components a) and b) (and optionally also c) and d)), the aqueous ultrasound coupling agent optionally comprises further conventional additives as known to the person skilled in the art.

For example, in one embodiment the aqueous ultrasound coupling agent comprises at least one pH-adjusting additive in order to adjust the pH of the agent to a level suitable for the intended clinical use. In one embodiment, the pH-adjusting additive is a metal hydroxide, preferably a group (I) metal hydroxide such as sodium hydroxide, potassium hydroxide or a mixture thereof. In a preferred embodiment, the pH-adjusting additive is sodium hydroxide. Alternatively, a base such as a tertiary amine (e.g. trimethylamine) can be added to the ultrasound coupling agent to adjust its pH. Optionally the aqueous ultrasound coupling agent (e.g. fluid) can be buffered, however the use of a buffer is not required.

As the solvent used to prepare the aqueous ultrasound coupling agent of the present invention, it is possible to use water or any conventional aqueous solution typically used for the purpose of preparing aqueous formulations for medical applications. For example, in one embodiment, the aqueous ultrasound coupling agent is prepared using saline, Ringer's solution, water-for-injection (WFI) or a mixture thereof. The aqueous ultrasound coupling agent according to the present invention may therefore comprise (in addition to components a)-d)) solvated ions of salts such as sodium chloride, potassium chloride, calcium chloride, sodium bicarbonate, and mixtures thereof. In general however it is desirable to keep the concentration of such ions in the ultrasound coupling agent low in order to maintain the “cleanliness” of the composition, which is important for the intended medical application. In one embodiment, the aqueous ultrasound coupling agent consists of components a) to d) as described here (with c) and d) being optional) and up to 10% (e.g. 0.01 to 10%) by weight of other components, preferably up to 5% by weight.

In one embodiment, the coupling agent may be sterilised by gamma irradiation and adjusted to pH 9-12, preferably pH 10-11 prior to gamma irradiation. This is believed to compensate for the observed drop in pH when the formulation is irradiated and/or stored. In another embodiment, the aqueous ultrasound coupling agent may have a pH of 6.0 to 9.0, preferably 6.5 to 8.0 after sterilisation by gamma irradiation and storage for at least 5 days. Most bacteria are neutrophils and grow best at near neutral pH, i.e. around 7. Where the pH of the formulation after manufacturing but before irradiation is above 9.0 this will reduce as much as possible the risk of bacterial growth occurring between mixing and sterilization. The reduction of pH (e.g. to 6.5 to 8.0) after sterilisation makes the formulation compatible for use without further adjustment.

In all aspects and embodiment of the present invention, where technically viable, the acoustic coupling agent may be a fluid or a gel. Correspondingly, the coupling agent may additionally comprise any appropriate gelling agent. Examples of gelling agents are well known in the art and include protein-based gelling agents such as gelatine, and polysaccharide-based gelling agents such as pectin, agar and alginates. Mixtures of gelling agents may be used.

### Properties

The aqueous ultrasound coupling agent general should have a pH which matches the pH of the tissue to which it is intended to be contacted with. Typically therefore, the aqueous ultrasound coupling agent has a pH in the range of 6.0 to 9.0, preferably 6.5 to 8.5, more preferably 6.5 to 8.0. In the case where the ultrasound coupling agent is intended to be used for invasive ultrasound imaging of brain tissue, a pH of 6.5 to 8.0 is especially preferred.

The skilled person knows that the pH of the composition may gradually change over time depending on storage conditions etc. For example, in general the pH of the composition directly after manufacture is higher than that of the composition after a period of prolonged storage. For the avoidance of doubt therefore, by the pH of the composition is meant the pH of the composition shortly before its intended application, as it is this that is important in determining the suitability of the composition for use on certain tissue types.

The amount of solute (including any dissolved ions) in the ultrasound coupling agent can be expressed as the osmotic concentration (also known as osmolarity) of the ultrasound coupling agent. In one embodiment, the ultrasound coupling agent has an osmotic concentration in the range of 200 to 500 mOsm/kg of aqueous ultrasound coupling agent, preferably 225 to 400 mOsm/kg, more preferably 250 to 350 mOsm/kg, such as 275 to 325 mOsm/kg. In an especially preferred embodiment, the ultrasound coupling agent has an osmotic concentration in the range of 280 to 300 mOsm/kg.

As discussed herein, the purpose of the ultrasound coupling agent of the present invention is not only to avoid air pockets between the ultrasound transducer/probe and tissue to be imaged and to facilitate a good acoustic coupling between the probe and tissue, but also to minimise the appearance of artefacts by matching the attenuation of the ultrasound coupling agent and that of the tissue to be imaged. The attenuation of a particular tissue/ultrasound coupling agent is however in part a function of the frequency of the ultrasound used for imaging (as well as the propagation length), and so a more common parameter used to determine the degree to which the ultrasound coupling agent is suitable for use with certain tissue types is the attenuation coefficient (α). This has units dB/(MHz·cm) and is a property of the material itself i.e. is not dependent on frequency or propagation length. The attenuation coefficient is measured by measuring the amplitude of signals obtained using pulse-echo measurements applied to the acoustic coupling agent of the present invention and comparing this to the amplitude using pulse-echo measurements applied to a specific reference material (e.g. water).

Typically the aqueous ultrasound coupling agent according to the present invention has an attenuation coefficient that is at least a factor of 30 larger than the attenuation coefficient for water, (i.e. α>0.066 dB/(MHz·cm)). In one embodiment, the attenuation coefficient is in the range of 0.10 to 1.10, preferably 0.15 to 1.00 dB/(MHz·cm), more preferably 0.20 to 0.90 dB/(MHz·cm). In one preferred embodiment, the attenuation coefficient is in the range of 0.30 to 0.80 dB/(MHz*cm). In an embodiment of the invention the coupling agent has an attenuation coefficient targeted at about 0.60 dB/(MHz cm) (±0.10 dB/MHz*cm), which is in the same order as the attenuation coefficient of the adult human brain.

In one embodiment, the ultrasound coupling agent has an attenuation coefficient between that of water (i.e. 0.022 dB/(MHz*cm)) and the attenuation coefficient of the target tissue (i.e. the tissue to be imaged). For example, when the ultrasound coupling agent is for use in invasive ultrasound imaging of the brain, the attenuation constant of the ultrasound coupling agent may be in the range of 0.022 to 0.60 dB/(MHz*cm), such as 0.10 to 0.60 dB/(MHz*cm), preferably 0.15 to 0.60 dB/(MHz*cm).

In all aspects described herein, the ultrasound coupling agent is preferably biocompatible, sterile and non-toxic.

In an additional aspect, the invention provides for a pre-filled syringe containing an acoustic coupling agent of any of the aspects and embodiments disclosed herein. A suitable syringe may be, for example, 10 to 250 ml in capacity, such as 25 to 100 ml in capacity. In one embodiment, the acoustic coupling agent is sterilised by gamma irradiation of the pre-filled syringe containing said coupling agent. Pre-filled syringes may optionally be provided with a filter to reduce the probability that larger droplets and any presence of larger particles in the ACA can enter the cavity during use. Typical filters will have a pore size around 0.1 to 5 m, preferably 0.8 to 1.5 m.

### Applications

In one aspect, the aqueous ultrasound coupling agent according to the present invention is for use in invasive ultrasound imaging of a subject. Examples of such uses include invasive ultrasound imaging during surgery, as well as non-surgical uses such as invasive diagnostic procedures (e.g. colorectal examination).

Various tissues/materials may be imaged using the ultrasound coupling agent of the present invention. In a preferred embodiment, the ultrasound coupling agent according to the present invention is for use in invasive ultrasound imaging of the brain, such as during brain surgery. In other embodiments, the ultrasound coupling agent according to the present invention is for use in invasive ultrasound imaging of other tissues such as heart tissue or breast tissue. The present inventors have established that the aqueous coupling agent according to the present invention has an attenuation coefficient which makes it particularly suitable for imaging of the heart i.e. which ultrasound coupling agent (e.g. fluid) generates fewer artefacts during invasive ultrasound imaging of the heart.

In another aspect, the present invention also provides a method of ultrasound imaging of a subject, comprising administering to the subject an ultrasound coupling agent according to the present invention through an opening in the body of the subject, applying ultrasound to said ultrasound coupling agent, and detecting ultrasound reflected from said ultrasound coupling agent. In one embodiment, the method is a method of ultrasound imaging of a subject during surgery, such as brain surgery, breast cancer surgery, or open-heart surgery. In another embodiment, the method is a method of ultrasound imaging of a subject during an invasive diagnostic procedure, such as colorectal examination.

In another aspect, the present invention provides an aqueous ultrasound coupling agent comprising:
a) 20.0 to 90.0 g/L of at least one triglyceride;
b) 1.2 to 5.4 g/L of at least one phospholipid;
c) optionally at least one humectant; and
d) optionally at least one non-ionic stabilising agent;
for use in invasive ultrasound imaging of the heart, such as during open-heart surgery, and wherein the attenuation coefficient (α) of the ultrasound coupling agent is in the range of 0.1 to 1.0 dB/(MHz·cm), preferably 0.1 to 0.8 dB(MHz·cm). Unexpectedly, when the aqueous ultrasound coupling agent having an attenuation coefficient within this range is used in invasive ultrasound imaging of the heart, the intensity and prevalence of image artefacts such as brightness artefacts is reduced. The nature and amount of each of components a)-d) in the aqueous ultrasound coupling agent of this aspect may be as recited for the aqueous ultrasound coupling agent of any other aspect described herein.

In another aspect, the present invention provides an aqueous ultrasound coupling agent comprising:
a) 20.0 to 90.0 g/L of at least one triglyceride;
b) 1.2 to 5.4 g/L of at least one phospholipid;
c) optionally at least one humectant; and
d) optionally at least one non-ionic stabilising agent;
for use in invasive ultrasound imaging of the breast, such as during breast cancer surgery, wherein the attenuation coefficient (α) of the agent is in the range of 0.1 to 1.2 dB/(MHz·cm), preferably 0.1 to 0.8 dB(MHz·cm). Unexpectedly, when the aqueous ultrasound coupling agent having an attenuation coefficient within this range is used in invasive ultrasound imaging of the breast, the intensity and prevalence of image artefacts such as brightness artefacts is reduced. The nature and amount of each of components a)-d) in the aqueous ultrasound coupling agent of this aspect may be as recited for the aqueous ultrasound coupling agent of any other aspect described herein.

In another aspect, the invention provides an aqueous ultrasound coupling agent as hereinbefore defined for use in invasive ultrasound imaging of the colon in colorectal cancer staging using a balloon stand-off system, wherein the attenuation coefficient (α) of the ultrasound coupling agent is in the range of 0.1 to 0.6 dB(MHz·cm). Unexpectedly, when the aqueous ultrasound coupling agent having an attenuation coefficient within this range is used in colorectal cancer staging using a balloon stand-off system, the intensity and prevalence of image artefacts such as brightness artefacts is reduced.

The balloon standoff may comprise an outer “balloon” sheath surrounding at least a part of an ultrasound transducer and a liquid or gel ultrasound coupling material between the ultrasound transducer and the sheath. In one embodiment, the volume of ultrasound coupling material between the ultrasound transducer and the sheath may be varied in order to allow ease of positioning and conform the shape of the balloon to the biological structure being examined. The ultrasound coupling material of this embodiment will be the aqueous ultrasound coupling agent as described herein in any appropriate aspect or embodiment. The material of the sheath may be any appropriate material, such as an elastomeric polymer material. In one embodiment the material of the sheath may be latex-free.

In one aspect, the ultrasound coupling agent is for use in the invasive ultrasound imaging of the brain, heart, breast, veins, or colon.

### Methods

In one aspect, the present invention provides a method of preparing an aqueous ultrasound coupling agent according to the present invention, or an aqueous ultrasound coupling agent for use according to the present invention, the method comprising:
i) providing a mixture comprising at least one triglyceride, at least one phospholipid and optionally at least one humectant; and
ii) diluting said mixture with water or an aqueous solution comprising water and optionally at least one non-ionic stabilising agent.

The mixture provided in step i) is preferably an aqueous solution of the at least one triglyceride, at least one phospholipid and optionally at least one humectant. Optionally further components such as a pH-adjusting additive can also be present in the mixture. As to the nature of the triglyceride, phospholipid and optional humectant components, these can be any of the options recited in earlier aspects. A mixture comprising soybean oil, lecithin, and optionally glycerol is particularly preferred.

In the second step, the mixture provided in the first step is diluted with water or an aqueous solution. The aqueous solution can be any conventional aqueous solution typically used for the purpose of preparing aqueous formulations for medical applications. For example, in one embodiment, the aqueous solution is saline, Ringer's solution, water-for-injection (WFI) or a mixture thereof.

The choice of diluent may in part be influenced by the type of tissue which is intended to be imaged. For example, where the ultrasound coupling agent is intended for use in the imaging of the heart, it may be convenient to use sterile water to dilute the mixture provided in step i). This is because when imaging the heart, the presence of ions is not important. On the other hand, when imaging the brain for example, the presence of sodium and chloride ions is important and so preferably saline is used.

Optionally, the aqueous solution further comprises at least one non-ionic stabilising agent as previously defined. The amount of water or aqueous solution used to dilute said mixture can be adjusted so as to achieve the desired concentrations of triglyceride, phospholipid and optional other components in the ultrasound coupling agent.

In one embodiment, any of the methods of the invention (e.g. above or below) include sterilisation of the acoustic coupling agent (of any embodiment disclosed herein). This may be by heat, gamma irradiation etc. Gamma irradiation is preferred, such as with a radiation dose of 10 to 100 kGy. In one embodiment, method comprises filling the acoustic coupling agent into a syringe and sterilising the acoustic coupling agent by gamma irradiation of a pre-filled syringe containing said coupling agent.

In another aspect, there is provided a method of formulating an aqueous ultrasound coupling agent according to the present invention, or an aqueous ultrasound coupling agent for use according to the present invention, the method comprising: i) providing a mixture comprising at least one triglyceride, at least one phospholipid and optionally at least one humectant;
ii) determining the attenuation coefficient (α) of the mixture;
iii) calculating the difference (d) between the attenuation coefficient of the mixture determined in step ii) and a reference attenuation coefficient (αᵣ);
iv) forming a diluted mixture by diluting the mixture provided in step i) with water or an aqueous solution comprising water and optionally at least one non-ionic stabilising agent, in an amount which is determined by a function f(d) of the difference (d) calculated in step iii); and
v) optionally repeating steps ii)-iv) on the diluted mixture obtained in step iv).

By starting with a concentrated mixture of at least one triglyceride, at least one phospholipid and optionally other components, it is possible to arrive at an aqueous ultrasound coupling agent (generally a fluid) having the desired attenuation coefficient i.e. the reference attenuation coefficient, simply by varying the amount of water or aqueous solution added to the mixture. Preferably the reference attenuation coefficient (αᵣ) corresponds to the attenuation coefficient of a target tissue in a subject to be imaged, such as brain tissue, heart tissue or breast tissue.

The value of these reference attenuation coefficients for any particular tissue may be found in published reference tables. The nature of the mixture/aqueous solution may be the same as in the method for preparing the aqueous ultrasound coupling agent according to the aspect previously described. Preferably the mixture comprises soybean oil, lecithin and glycerol.

Regarding the function f(d) used to determine the amount of water/aqueous solution to be added to the mixture to form the diluted mixture, this may be determined experimentally, e.g. using calibration curves etc. In one embodiment, the function is a linear function i.e. represented by the equation:

ƒ(d)=kd+c

wherein k and c are constants. The value of the constant k and c can be determined experimentally.

In another aspect, the invention provides a kit comprising:
A) a mixture comprising at least one triglyceride, at least one phospholipid and optionally at least one humectant; and
B) water or an aqueous solution comprising water and optionally at least one non-ionic stabilising agent.

The mixture and aqueous solution are preferably as defined in any aspect described herein. Advantageously, the kit is suitable for carrying out the method for preparing or method for formulating the aqueous ultrasound coupling agent as defined herein.

Suitable kits may include a syringe containing component A), which may be suitable for adding a selected quantity of component a) to the diluent component B).

As used herein, amounts which are indicated as “about”, “approximately”, etc may be the exact value stated or may vary by ±10%, preferably ±5% or ±1%. The same applies to the ends of ranges. Formulations “comprising” certain components may also “consist essentially” of such components or consist solely of such components. Similarly, compositions “consisting essentially of” listed components may consist solely of such components or may contain 105<preferably 5% or 1% of other materials. All % are by weight where context allows, unless indicated otherwise.

### Examples

### General Experimental

Acoustic Contact Agent (ACA) was formulated from a concentrate of lipid components and humectant (e.g. glycerol) where present as an emulsion in purified (DI) water. The concentrate was initially formulated at around 20 mg triglyceride per ml. The concentrate was then diluted to the appropriate concentration with an aqueous fluid. Aqueous fluids used include purified water (deionised—DI) or saline (e.g. 0.9% NaCl). Where present, stabiliser such as Polysorbate 80 (P80, Tween 80) was added to the aqueous component. pH was adjusted with NaOH as necessary.

### Lipid Concentrate (LC)

The Lipid Concentrate (LC) used was typically an emulsion of 20% Soybean Oil, 1.2% Egg Yolk Phospholipids, 2.25% Glycerine, and Water for Injection. pH was adjusted with sodium hydroxide to around pH 8.

### Materials, Test Samples and Measurements

Samples were stored at room temperature, with no direct exposure to sunlight. Visual analysis was done by observation of the syringe prior to extraction for further analysis, without any agitation of the samples which might cause redispersion or temporary mixing. For every measurement point, 5 ml of the emulsion was squeezed from each syringe after visual analysis. The 5 ml aliquots were subsequently used for all analyses of a specific time point. The autoclave (CertoClav® CVEL 12 L) temperature was controlled manually via a pressure gauge, and the duration of each cycle was controlled via a timer. The conditions used were 20 minutes at 121° C.

### Measurements

pH measurements were collected using a Mettler Toledo SevenEasy pH-meter calibrated with AVS TITRINORM Buffer solution pH 4 and AVS TITRINORM Buffer solution pH 7, and controlled with AVS TITRJNORM Buffer solution pH 5 as part of a daily routine.

Dynamic light scattering measurements were collected using a Malvern Zetasizer Nano Series Nano-ZS. Here, the sample was diluted by a factor 100 in DI water immediately prior to analysis. For size control and calibration, a Nanosphere size Standard 203±5 nm was used. This is made fresh and run every day the measurement was done. Average droplet size reported from ZetaSizer measurements is the Z-average diameter.

The microscope used is an Olympos BX43 equipped with an Olympus XM10 camera with pre-calibrated measurements (embedded in images). Osmolality measurements were made using an OSMOMAT 030-D-D3P from GonoTec, Ser. No. 08 02 22. Here, 50 microliter of the sample was pipetted into the measuring vessel, making sure to avoid any formation of air bubbles. Prior to sample analysis, the instrument was calibrated with a GonoTec calibration standard with an expected value of 300 mOsmol/kg NaCl/H20 (acceptable range 298-302 mOsmol/kg). The calibration standard was run in triplicate.

### Example 1—Emulsion Stability and Sterilization Method

Tests on samples of ACA consisting of 35% Lipid Concentrate (see above—at 200 mg/ml triglyceride) and 65% NaCl (0.9%) showed that the diluted fat emulsion phase separated after autoclaving. Lipid Concentrate diluted with sterile water did not phase separate but was less desirable due to the non-isotonic and non-ionic properties that could be a potential safety concern. Therefore, irradiation was tested as alternative means of sterilizing the device.

The results from gamma radiated samples (all containing 35% LC, 65% aqueous component) showed that the sample with Ringer solution, glycerol (26 ml 85% glycerol to 1000 ml) and added Tween 80 (1% of saline volume), the sample with saline and added Tween 80 (1%) and the sample R-16 with saline, Tween 80 (1%) and added glycerol did not have any phase separation, discolouring or precipitate. Use of Tween 80 thus appeared to offer an advantage for stable storage, particularly when gamma-irradiation is used for sterilisation.

### Example 2—Visual Analysis of ACA Storage

The Lipid Concentrate (LC) described above was diluted with sterile water or 0.9% saline with and without polysorbate 80 (tween80). The formulations were all gamma-irradiated, stored for some weeks and assessed visually.

TABLE 1
Test samples for phase separation.

| | | | | Meas. |
|---|---|---|---|---|
| | | | | time |
| | | Storage | | (weeks |
| Sample | | temp. | | after |
| no | ACA-dilution | [° C.] | Measurements | received) |
| S10-1 | 17.5 ml LC + | 20 | Visual | 0/2/4/6/8 |
| | 32.5 ml saline. | | | |
| | pH adjusted with NaOH | | | |
| S10-2 | 17.5 ml LC + | 20 | Visual | 0/2/4/6/8 |
| | 32.5 ml saline. | | | |
| | pH adjusted with NaOH | | | |
| S10-3 | 17.5 ml LC + | 40 | Visual | 0/2/4/6/8 |
| | 32.5 ml saline. | | | |
| | pH adjusted with NaOH | | | |
| S10-4 | 17.5 ml LC + | 40 | Visual | 0/2/4/6/8 |
| | 32.5 ml saline. | | | |
| | pH adjusted with NaOH | | | |
| S10-5 | 17.5 ml LC + 32.5 ml | 20 | Visual | 0/2/4/6/8 |
| | saline and TWEEN 80 | | | |
| | (10/1000) dilution. | | | |
| | pH adjusted with NaOH | | | |
| S10-6 | 17.5 ml LC + 32.5 ml | 20 | Visual | 0/2/4/6/8 |
| | saline and TWEEN 80 | | | |
| | (10/1000) dilution. | | | |
| | pH adjusted with NaOH | | | |
| S10-7 | 17.5 ml LC + 32.5 ml | 40 | Visual | 0/2/4/6/8 |
| | saline and TWEEN 80 | | | |
| | (10/1000) dilution. | | | |
| | pH adjusted with NaOH | | | |
| S10-8 | 17.5 ml LC + 32.5 ml | 40 | Visual | 0/2/4/6/8 |
| | saline and TWEEN 80 | | | |
| | (10/1000) dilution. | | | |
| | pH adjusted with NaOH | | | |
| S10-9 | 17.5 ml LC + 32.5 ml | 20 | Visual | 0/2/4/6/8 |
| | saline and TWEEN 80 | | | |
| | (3/1000) dilution. | | | |
| | pH adjusted with NaOH | | | |
| S10-10 | 17.5 ml LC + 32.5 ml | 20 | Visual | 0/2/4/6/8 |
| | saline and TWEEN 80 | | | |
| | (3/1000) dilution. | | | |
| | pH adjusted with NaOH | | | |
| S10-11 | 17.5 ml LC + 32.5 ml | 40 | Visual | 0/2/4/6/8 |
| | saline and TWEEN 80 | | | |
| | (3/1000) dilution. | | | |
| | pH adjusted with NaOH | | | |
| S10-12 | 17.5 ml LC + 32.5 ml | 40 | Visual | 0/2/4/6/8 |
| | saline and TWEEN 80 | | | |
| | (3/1000) dilution. | | | |
| | pH adjusted with NaOH | | | |
| S10-13 | 17.5 ml LC + 32.5 ml | 20 | Visual | 0/2/4/6/8 |
| | sterile water dilution. | | | |
| S10-14 | 17.5 ml LC + 32.5 ml | 20 | Visual | 0/2/4/6/8 |
| | sterile water dilution. | | | |
| S10-15 | 17.5 ml LC + 32.5 ml | 40 | Visual | 0/2/4/6/8 |
| | sterile water dilution. | | | |
| S10-16 | 17.5 ml LC + 32.5 ml | 40 | Visual | 0/2/4/6/8 |
| | sterile water dilution. | | | |
| S10-17 | 17.5 ml LC + 32.5 ml | 20 | Visual | 0/2/4/6/8 |
| | sterile water and | | | |
| | glycerol dilution. | | | |
| S10-18 | 17.5 ml LC + 32.5 ml | 20 | Visual | 0/2/4/6/8 |
| | sterile water and | | | |
| | glycerol dilution. | | | |
| S10-19 | 17.5 ml LC + 32.5 ml | 40 | Visual | 0/2/4/6/8 |
| | sterile water and | | | |
| | glycerol dilution. | | | |
| S10-20 | 17.5 ml LC + 32.5 ml | 40 | Visual | 0/2/4/6/8 |
| | sterile water and | | | |
| | glycerol dilution. | | | |

The formulations diluted with purified water were found to be stable to storage. However, the formulations diluted with saline tended to phase-separate unless TWEEN 80 was added. The addition of the stabiliser improved the storage stability.

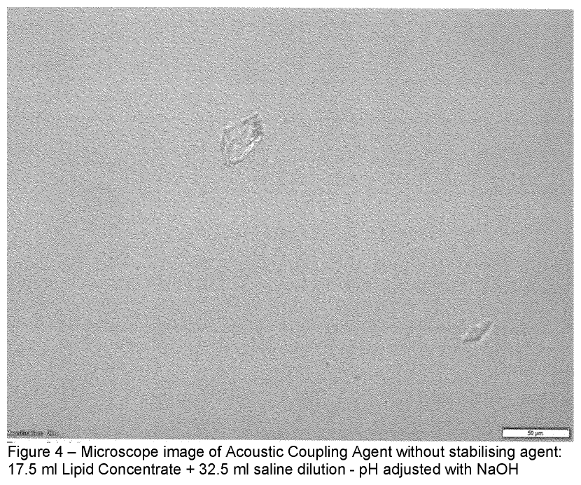

FIG. 4 shows a microscope image of a formulation of 17.5 ml Lipid Concentrate+32.5 ml saline dilution, pH adjusted with NaOH. Particles are visible in the formulation in the absence of stabilising agent.

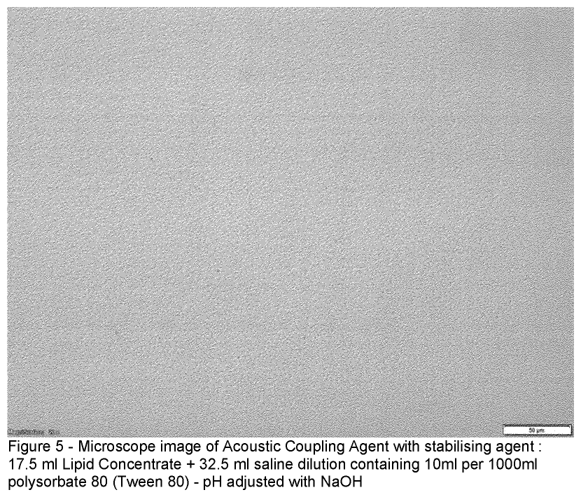

FIG. 5 shows a microscope image of a formulation of 17.5 ml Lipid Concentrate+32.5 ml saline dilution containing 10 ml per 1000 ml polysorbate 80 (Tween 80). The formulation is pH adjusted with NaOH. With the stabilising agent included, no particles are visible.

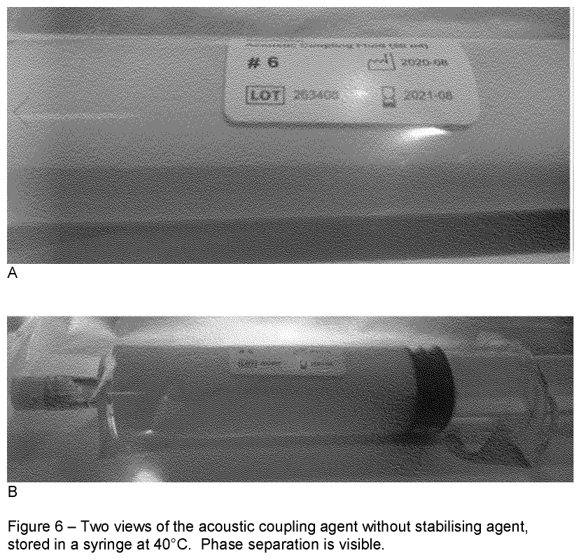

FIG. 6 shows a syringe containing the acoustic coupling agent formulated without stabilising agent, stored at 40° C. Separation into an opaque phase at the top and a more translucent phase at the bottom can be seen.

### Example 3—pH Stability

Samples with LC and 1% w/v Tween 80 (polysorbate 80) in pH-adjusted saline were compared to the corresponding pH evolution of reference systems (saline and Lipid Concentrate (LC) diluted with saline). The samples will be prepared in glass vials on day 1 and measured for three consecutive days without any transfer between sample compartments in order to avoid any artefacts emanating from e.g. leachables.

TABLE 2
pH stability

| | | pH | pH | pH | pH |
|---|---|---|---|---|---|
| Sample | Description | Day1 | Day 2 | Day 3 | Change |
| A | 0.7 wt % NaCl, pH 10.45 | 10.45 | 10.26 | 10.07 | 0.38 |
| B | 0.7 wt % NaCl, pH 10.45 | 10.45 | 10.11 | 10.01 | 0.44 |
| C | 0.7 wt % NaCl, pH 10.45 | 10.45 | 9.99 | 9.88 | 0.57 |
| D | LC (35%) diluted with | 7.84 | 7.78 | 7.64 | 2.81 |
| | 0.7 wt % NaCl, | | | | |
| | pH 10.45 (65%) | | | | |
| E | LC (35%) diluted | 7.85 | 7.95 | 7.88 | 2.57 |
| | with 0.7 wt % NaCl, | | | | |
| | pH 10.45 (65%) | | | | |
| F | LC (35%) diluted | 7.90 | 7.94 | 7.90 | 2.55 |
| | with 0.7 wt % NaCl, | | | | |
| | pH 10.45 (65%) | | | | |
| G | LC (35%) diluted | 7.60 | 7.56 | 7.47 | 2.98 |
| | with 65% 0.7 wt % | | | | |
| | NaCl containing 1% | | | | |
| | Tween 80, pH 10.45 | | | | |
| H | LC (35%) diluted | 7.61 | 7.57 | 7.52 | 2.93 |
| | with 65% 0.7 wt % | | | | |
| | NaCl containing 1% | | | | |
| | Tween 80, pH 10.45 | | | | |
| I | LC (35%) diluted | 7.61 | 7.58 | 7.54 | 2.91 |
| | with 65% 0.7 wt % | | | | |
| | NaCl containing 1% | | | | |
| | Tween 80, pH 10.45 | | | | |

It is notable that without Tween 80 (Samples D-F), the change in pH encompassed the range 2.55 to 2.81 pH units, a variation of 0.67 units. When Tween 80 was included (Samples G-I), the variation was much smaller, covering the range 2.91 to 2.98 pH units (a variation of 0.17 units). This was comparable to the control sample containing only pH adjusted saline solution (variation of 0.19 units). It therefore appears that variation of pH is more predictable when Tween is included.

### Example 4—Attenuation Coefficient α for Different Concentrations of ACA

The speed of sound c and attenuation coefficient α of the acoustic coupling agent (ACA) has been measured in several measurements performed at the ultrasound laboratory. The measurements have covered the frequency range between 3-12 MHz, which should be representative for ultrasound imaging in surgery. In one study the speed of sound and attenuation was measured for several concentrations of ACA. The ACA formulations were generated by dilution of the Lipid Concentrate (LC) described above with 0.9% sodium chloride solution in water.

TABLE 3
Attenuation coefficient α for varying concentrations
of Lipid Concentrate (LC) in the acoustic coupling agent (ACA).

| | | | | α | average |
|---|---|---|---|---|---|
| | | | | [dB/(MHz | α[dB/ |
| | Concentration | Temp | Frequency | cm)] ± | (MHz |
| | of ACA | [° C.] | [MHz] | stdev | cm)] |
| ACA I | 30.0% LC | 23.2 | 3 | 0.559 ± 0.012 | 0.526 |
| | 200 mg/ml | | 5 | 0.534 ± 0.008 | |
| | 70.0% NaCl | | 9 | 0.518 ± 0.007 | |
| | (9 mg/ml) | | 12 | 0.491 ± 0.012 | |
| ACA II | 32.5% LC | 23.2 | 3 | 0.606 ± 0.011 | 0.575 |
| | 200 mg/ml | | 5 | 0.582 ± 0.005 | |
| | 67.5% NaCl | | 9 | 0.567 ± 0.006 | |
| | (9 mg/ml) | | 12 | 0.544 ± 0.009 | |
| ACA III | 35.0% LC | 23.4 | 3 | 0.651 ± 0.010 | 0.620 |
| | 200 mg/ml | | 5 | 0.631 ± 0.007 | |
| | 65.0% NaCl | | 9 | 0.613 ± 0.007 | |
| | (9 mg/ml) | | 12 | 0.583 ± 0.013 | |
| ACA IV | 37.5% LC | 23.6 | 3 | 0.681 ± 0.014 | 0.653 |
| | 200 mg/ml | | 5 | 0.661 ± 0.008 | |
| | 62.5% NaCl | | 9 | 0.645 ± 0.008 | |
| | (9 mg/ml) | | 12 | 0.625 ± 0.017 | |
| ACA V | 40.0% LC | 23.3 | 3 | 0.714 ± 0.010 | 0.691 |
| | 200 mg/ml | | 5 | 0.701 ± 0.005 | |
| | 60.0% NaCl | | 9 | 0.686 ± 0.008 | |
| | (9 mg/ml) | | 12 | 0.662 ± 0.024 | |

The speed of sound measured at room temperature is approximately 1497 m/s. Measurements of attenuation for different concentrations of ACA is provided in Table 3. The acoustic coupling agent does not provide any scattering of sound, and hence the attenuation of sound is caused by absorption.

1. An aqueous ultrasound coupling agent comprising:
a) 20.0 to 90.0 g/L of at least one triglyceride;
b) 1.2 to 5.4 g/L of at least one phospholipid;
c) optionally at least one humectant; and
d) optionally at least one non-ionic stabilising agent.

2. An aqueous ultrasound coupling agent according to claim 1, wherein component a) is present in an amount of 40.0 to 90.0 g/L, preferably 50.0 to 90.0 g/L, more preferably 55.0 to 85.0 g/L, most preferably 60.0 to 80.0 g/L.

3. An aqueous ultrasound coupling agent according to claim 1 or 2, wherein component b) is present in an amount of 2.5 to 5.2 g/L, preferably 3.0 to 5.0 g/L, more preferably 3.5 to 4.8 g/L, most preferably 3.8 to 4.5 g/L.

4. An aqueous ultrasound coupling agent according to any preceding claim, wherein the ratio by weight between component a) and component b) is in the range of 8:1 to 25:1, preferably 10:1 to 22:1, more preferably 12:1 to 20:1, most preferably 14:1 to 18:1.

5. An aqueous ultrasound coupling agent according to any preceding claim, wherein said triglyceride is selected from vegetable oils or animal fats, preferably wherein said vegetable oil or animal fat is selected from the group consisting of soybean oil, olive oil, palm oil, copra oil, milk fats, fish oils, fish liver oils and mixtures thereof, more preferably wherein said triglyceride is soybean oil.

6. An aqueous ultrasound coupling agent according to any preceding claim, wherein component b) is lecithin, preferably egg yolk lecithin, soy lecithin or a mixture thereof.

7. An aqueous ultrasound coupling agent according to any preceding claim, wherein the humectant is present in an amount of 2.2 to 9.5 g/L, preferably 4.5 to 9.2 g/L, more preferably 5.5 to 9.0 g/L, most preferably 6.5 to 8.5 g/L.

8. An aqueous ultrasound coupling agent according to any preceding claim, wherein the humectant is selected from the group consisting of polyols (such as glycerol, propylene glycol and sorbitol), lactic acid and mixtures thereof, preferably wherein the humectant is glycerol.

9. An aqueous ultrasound coupling agent according to any preceding claim, wherein the non-ionic stabilising agent is present in an amount of 2.0 to 9.0 g/L, preferably 4.0 to 8.8 g/L, more preferably 6.0 to 8.5 g/L, most preferably 6.0 to 8.0 g/L.

10. An aqueous ultrasound coupling agent according to any preceding claim, wherein the non-ionic stabilising agent has a hydrophilic-lipophilic balance (HLB) value in the range of 8-14.

11. An aqueous ultrasound coupling agent according to any preceding claim wherein the non-ionic stabilising agent is a polyoxyethylene-based stabilising agent such as a polysorbate, poloxamer or a mixture thereof.

12. An aqueous ultrasound coupling agent according to any preceding claim, wherein the non-ionic stabilising agent is a polysorbate, preferably selected from the group consisting of polysorbate 80, polysorbate 60, polysorbate 40, polysorbate 20 and mixtures thereof, more preferably wherein the stabilising agent is polysorbate 80.

13. An aqueous ultrasound coupling agent according to any preceding claim, further comprising at least one pH-adjusting additive, preferably wherein the pH-adjusting additive is a metal hydroxide, more preferably wherein the pH-adjusting additive is selected from sodium hydroxide, potassium hydroxide or mixtures thereof, most preferably wherein the pH-adjusting additive is sodium hydroxide.

14. An aqueous ultrasound coupling agent according to any preceding claim, having a pH in the range of 6.0 to 9.0, preferably 6.5 to 8.5, more preferably 6.5 to 8.0.

15. An aqueous ultrasound coupling agent according to any preceding claim, having an attenuation coefficient (α) greater than 0.066 dB/(MHz·cm), preferably wherein the attenuation coefficient (α) is in the range 0.20 to 1.10 dB/(MHz·cm), more preferably 0.50 to 1.00 dB/(MHz·cm), most preferably 0.70 to 0.90 dB/(MHz·cm).

16. An aqueous ultrasound coupling agent according to any preceding claim comprising:
a) 60.0 to 80.0 g/L soybean oil;
b) 3.8 to 4.5 g/L lecithin;
c) optionally 6.5 to 8.5 g/L glycerol; and
d) optionally 6.0 to 8.0 g/L polysorbate 80.

17. An aqueous ultrasound coupling agent according to any preceding claim for use in invasive ultrasound imaging of a subject, such as during surgery or during an invasive diagnostic procedure.

18. An aqueous ultrasound coupling agent for use according to claim 17, wherein the invasive ultrasound imaging is invasive ultrasound imaging of the brain, heart, breast, veins, or colon.

19. An aqueous ultrasound coupling agent according to any of claims 1 to 16 for use in invasive ultrasound imaging of the heart, such as during open-heart surgery, wherein the attenuation coefficient (α) of the ultrasound coupling agent is in the range of 0.1 to 1.0 dB/(MHz·cm), preferably 0.1 to 0.8 dB(MHz·cm).

20. An aqueous ultrasound coupling agent according to any of claims 1 to 16 for use in invasive ultrasound imaging of the breast, such as during breast cancer surgery, wherein the attenuation coefficient (α) of the agent is in the range of 0.1 to 1.2 dB/(MHz·cm), preferably 0.1 to 0.8 dB/(MHz·cm).

21. An aqueous ultrasound coupling agent according to any of claims 1 to 16 for use in invasive ultrasound imaging of the colon in colorectal cancer staging using a balloon stand-off system, wherein the attenuation coefficient (α) of the ultrasound coupling agent is in the range of 0.1 to 0.6 dB(MHz·cm).

22. A method of preparing an aqueous ultrasound coupling agent according to any of claims 1 to 16, or an aqueous ultrasound coupling agent for use according to any of claims 17 to 21, the method comprising:
i) providing a mixture comprising at least one triglyceride, at least one phospholipid and optionally at least one humectant; and
ii) diluting said mixture with water or an aqueous solution comprising water and optionally at least one non-ionic stabilising agent.

23. A method of formulating an aqueous ultrasound coupling agent according to any of claims 1 to 16, or an aqueous ultrasound coupling agent for use according to any of claims 17 to 21, the method comprising:
i) providing a mixture comprising at least one triglyceride, at least one phospholipid and optionally at least one humectant;
ii) determining the attenuation coefficient (α) of the mixture;
iii) calculating the difference (d) between the attenuation coefficient of the mixture determined in step ii) and a reference attenuation coefficient (αᵣ);
iv) forming a diluted mixture by diluting the mixture provided in step i) with water or an aqueous solution comprising water and optionally at least one non-ionic stabilising agent, in an amount which is determined by a function f(d) of the difference (d) calculated in step iii); and
v) optionally repeating steps ii)-iv) on the diluted mixture obtained in step iv).

24. A method according to claim 23, wherein the function f(d) is represented by the equation:
ƒ(d)=kd+c
wherein k and c are constants;
preferably wherein the reference attenuation coefficient (αᵣ) corresponds to the attenuation coefficient of a target tissue in a subject, such as brain tissue, heart tissue or breast tissue.

25. A kit comprising:
A) a mixture comprising at least one triglyceride, at least one phospholipid and optionally at least one humectant; and
B) water or an aqueous solution comprising water and optionally at least one non-ionic stabilising agent.
